(19) United States
(12) Patent Application Publication
(10) Pub. No.: US 2026/0367053 A1
(43) Pub. Date: Nov. 19, 2026

(54) INTELLIGENT PLACEMENT OF GAS COLLECTION SYSTEMS FOR LANDFILL GAS PRODUCTION

(71) Applicant: SeekOps Inc., Austin, TX (US)

(72) Inventors: Iain Cooper, Canyon Lake, TX (US); Brendan James Smith, Lakeway, TX (US); Robert Russell, Cedar Park, TX (US); Kyle Dawson, Austin, TX (US); Evangeline Kyle, Austin, TX (US); Gregorio Nunez, Pflugerville, TX (US)

(21) Appl. No.: 19/148,257

(22) PCT Filed: Jan. 13, 2024
(86) PCT No.: PCT/US24/11529

§ 371 Date: Jul. 15, 2025

Related U.S. Application Data
(60) Provisional application No. 63/439,133, filed on Jan. 15, 2023.

(51) Int. Cl. G01N 33/00; G05D 1/229; G05D 1/246; G05D 1/628; G05D 105/80; G05D 107/90; G05D 109/20; G05D 111/50

(52) U.S. Cl. CPC G01N 33/0047; G01N 33/004; G01N 33/0067; G05D 1/229; G05D 1/246; G05D 1/628; G05D 2105/80; G05D 2107/90; G05D 2109/20; G05D 2111/56

(57) ABSTRACT

Please replace the Abstract with the following amended Abstract:

Systems, devices, and methods for determining a placement of a gas collection well based on gathered trace gas data and/or wind speed data including to achieve enhanced gas production: gathering trace gas data over an area of interest; generating at least one of: a heat map and a topographical map of gas concentration data based on the gathered trace gas data of the area of interest; determining at least one of: a local maxima and a global maximum of increased gas concentration; and placing one or more gas collection well proximal to the determined at least one of: the local maxima and the global maximum.

### CROSS-REFERENCE TO RELATED APPLICATIONS

This application is a 35 U.S. C § 371 National Stage Entry of International Application No. PCT/US 2024/011529, filed Jan. 13, 2024, which claims the priority benefit of U.S. Provisional Patent Application Ser. No. 63/439,133, filed Jan. 15, 2023, all of which are hereby incorporated herein by reference in their entireties for all purposes.

### FIELD OF ENDEAVOR

The invention relates generally to gas collection, and more particularly to gas collection at landfills.

### BACKGROUND

Methane (CH4) is an odorless and colorless trace gas including naturally occurring organic molecules, present in the atmosphere at average ambient levels of approximately 1.85 ppm as of 2018, and is projected to continually increase in quantity. While methane is found globally in the atmosphere, a significant amount is collected or “produced” through anthropogenic processes including exploration, extraction, and distribution of petroleum in the form of natural gas. Natural gas, an odorless and colorless gas, is a primary source of energy used to produce electricity and heat. The main component of natural gas is methane (93.9 mol % CH4 typ.). While extraction of natural gas is a large source of methane released into the atmosphere, major contributors of methane also include livestock farming (enteric fermentation), and solid waste and wastewater treatment (anaerobic digestion).

### SUMMARY

A method embodiment for determining a placement of a gas collection well based on gathered trace gas data may include: gathering trace gas data over an area of interest; generating at least one of: a heat map and a topographical map of gas concentration data based on the gathered trace gas data of the area of interest; determining at least one of: a local maxima and a global maximum of increased gas concentration; and placing one or more gas collection well proximal to the determined at least one of: the local maxima and the global maximum.

Additional method embodiments may include: gathering trace gas data over one or more previously installed gas collection wells. Additional method embodiments may include: gathering trace gas data from a working face of the area of interest to ensure that the generated heat map may be more representative of a subsurface gas distribution. Additional method embodiments may include: gathering trace gas data from an active waste burial region to ensure that the generated heat map may be more representative of a subsurface gas distribution.

Additional method embodiments may include: placing one or more unmanned aerial system (UAS) waypoints over the area of interest. Additional method embodiments may include: repeating one or more flights of the UAS over the area of interest using the placed one or more UAS waypoints to ensure that repeat flights following a consistent path.

Additional method embodiments may include: utilizing a UAS system terrain following capability to ensure that a consistent distance may be kept as near as possible to a surface of the area of interest.

Another method embodiment for determining a placement of a gas collection well based on gathered trace gas data and wind speed may include: gathering trace gas data and wind speed over an area of interest; calculating a point-wide mass flow rate of a trace gas over the area of interest; generating at least one of: a heat map and a topographical map of the mass flow rate data over the area of interest; determining at least one of: a local maxima and a global maximum of increased mass flow rate; and placing one or more gas collection wells proximal to the determined at least one of: the local maxima and the global maximum.

Additional method embodiments may include: gathering trace gas data over one or more previously installed gas collection wells. Additional method embodiments may include: gathering trace gas data from a working face of the area of interest to ensure that the generated heat map may be more representative of a subsurface gas distribution. Additional method embodiments may include: gathering trace gas data from an active waste burial region to ensure that the generated heat map may be more representative of a subsurface gas distribution.

Additional method embodiments may include: placing one or more unmanned aerial system (UAS) waypoints over the area of interest. Additional method embodiments may include: repeating one or more flights of the UAS over the area of interest using the placed one or more UAS waypoints to ensure that repeat flights following a consistent path.

Additional method embodiments may include: utilizing a UAS system terrain following capability to ensure that a consistent distance may be kept as near as possible to a surface of the area of interest.

A system embodiment may include: a processor having addressable memory, where the processor may be configured to: determine a flight path for an unmanned aerial vehicle (UAV) over at least a portion of a landfill; receive trace gas emissions, via a gas sensor of the UAV, in the region of the flight path; receive wind measurements corresponding to the received trace gas emissions; determine one or more regions of elevated trace gas emissions in the landfill based on: the received trace gas emissions, the received wind measurements, and an elevation of the UAV above a ground surface of the landfill during the flight path; and determine a placement of one or more gas collection wells in the landfill based on the determined one or more regions of elevated trace gas emissions.

In additional system embodiments, the gas sensor may be configured to detect one or more of: methane and carbon dioxide. In additional system embodiments, the elevation of the UAV above the ground surface of the landfill may be kept at a consistent distance via a terrain following of the UAV. In additional system embodiments, the elevation of the UAV above the ground surface of the landfill may be between 1 meter and 10 meters.

In additional system embodiments, the processor may be further configured to repeat the determined flight path for the UAV (UAV) to determine any changes in the determine one or more regions of elevated trace gas emissions in the landfill over time. In additional system embodiments, the received wind measurements may be via an anemometer on the UAV.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts a landfill site, according to one embodiment;

FIG. 2 depicts a walking survey of methane concentrations above landfill waste-in-place region, according to one embodiment;

FIG. 3 depicts an aerial mapping survey of the landfill tilted away from, a top-down view such that the variation in the surface elevation can be seen, according to one embodiment;

FIG. 4 depicts a landfill site with the different areas identified, according to one embodiment;

FIG. 5 depicts a GPS track of the aircraft at a landfill site, according to one embodiment;

FIG. 6 depicts a dense survey of a landfill showing high concentration regions resulting from a UAS-deployed methane concentration sensor and the outline of the region of the landfill site, according to one embodiment;

FIG. 7 depicts a top-down view of measurement methane enhancements across the landfill surface, according to one embodiment;

FIG. 8 depicts a windrose showing wind variability during the methane measurements, according to one embodiment;

FIG. 9 depicts a side view of methane enhancements across the surface of the landfill, according to one embodiment;

FIG. 10 depicts an initial placement of gas collection wells for plumbing into gas upgrading system with locations shown by an ‘X’, according to one embodiment;

FIG. 11 depicts a high level flowchart of a method embodiment for determining a placement of a gas collection well based on gathered trace gas data, according to one embodiment;

FIG. 12 depicts a high level flowchart of a method embodiment for determining a placement of a gas collection well based on gathered trace gas data and wind speed, according to one embodiment;

FIG. 13 illustrates an example top-level functional block diagram of a computing device embodiment;

FIG. 14 shows a high-level block diagram and process of a computing system for implementing an embodiment of the system and process;

FIG. 15 shows a block diagram and process of an exemplary system in which an embodiment may be implemented;

FIG. 16 depicts a cloud computing environment for implementing an embodiment of the system and process disclosed herein;

FIG. 17 depicts a system for detecting trace gases, according to one embodiment;

FIG. 18 depicts a field of view of an unmanned aerial system (UAS) flying over a landfill, according to one embodiment;

FIG. 19 depicts the UAS of FIG. 18 flying over the landfill with gas collection wells installed, according to one embodiment; and

FIG. 20 depicts a high-level block diagram of an unmanned aerial system (UAS) trace gas detection system, according to one embodiment.

### DETAILED DESCRIPTION

The following description is made for the purpose of illustrating the general principles of the embodiments disclosed herein and is not meant to limit the concepts disclosed herein. Further, particular features described herein can be used in combination with other described features in each of the various possible combinations and permutations. Unless otherwise specifically defined herein, all terms are to be given their broadest possible interpretation including meanings implied from the description as well as meanings understood by those skilled in the art and/or as defined in dictionaries, treatises, etc.

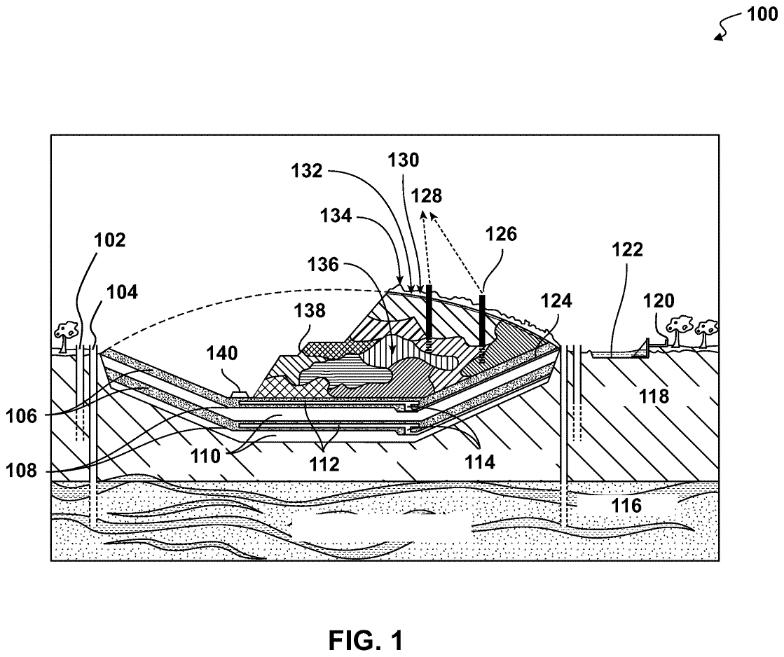

Modern landfill sites are ever-evolving topologies where waste is buried and capped. There are typically two elements to a landfill site 100 as shown in FIG. 1. The first element of the landfill site 100 is the waste-in-place (WIP) which is the previously buried refuse that has been capped, typically with a clay and or synthetic cap and then covered with vegetation/grass. An additional benefit of the unmanned aerial system (UAS) approach disclosed herein relates to preparation of the vegetation ahead of emissions surveys. The second element of the landfill site 100 is the active burial region and the working face, which is where waste is actively buried, typically on a dawn to dusk operational schedule, and with that every changing topography.

The landfill site 100 may include a gas monitoring probe 102; a ground water monitoring well 104; a drainage layer 106; a synthetic liner 108; a compacted clay liner 110; a perforated leachate collection pipe 112; a leachate collection sump with riser 114; ground water 116; existing ground 118; a stormwater outlet 120; a rainwater retention pond 122; leachate to treatment plant 124; a gas collection well 126; landfill gas to flare station or to energy utilization plant 128; a moisture barrier layer 130; granular drainage material 132; final clay and synthetic cap with vegetation 134; in place refuse 136; working face 138; and a storm water control berm 140.

The disclosed system and method relate to the WIP region. As part of the WIP region, to allow for the natural degradation of the waste, gas collection systems are positioned in wells that are drilled through the cap and into the WIP region to assist with the collection of trace gases such as methane, carbon dioxide, and other naturally occurring by-products of the solid waste decay process. These landfill gas collection wells are then routed to a central facility that either flares the waste gas or the waste gas is sent to on-site upgrading facilities that will purify the gas, typically stripping out hydrogen sulfide (H₂S) and other contaminants so that the gas can either be used for local heat or power, or can be further upgraded to the supply pipeline. The disclosed system and method may be used with any subsurface that may have trace gas in it, which is not limited to the WIP region of landfills.

With more landfill companies looking to be part of the circular economy and monetize their waste gas streams, it is essential that the process by which this gas is collected is cost-effective and optimal in terms of maximizing the gas that is processed.

The gas detection system and method described herein may use a Tunable Diode Laser Absorption Spectrometer tuned specifically to highly sensitive detection of methane, carbon dioxide, and/or other trace gases that is deployed on an unmanned aerial system (UAS) or drone that yields a highly flexible deployment over the surface of the landfill site, enabling detailed coverage of methane gas enhancements above the elevated background levels of methane that are typical at a landfill site 100. The drone also enables the active burial of waste to continue without interruption and enables any capping that may be performed to continue too. The unobtrusiveness of the system is one element that is key to optimal efficiency in terms of well placement.

One method for positioning the placement of gas collection wells is to drill test wells and sample methane concentrations in the subsurface of the landfill in those wells at numerous sites across the WIP section. Additionally, tools are subsequently run into these wells that can measure the in-situ concentration of methane in the subsurface. These would be run at an additional expense, and the results take additional time to process, and would then only yield an indication of the methane distribution and not a dense map as is achieved with the high-frequency measurements on the drone, This approach may be time-consuming, costly and given the equipment that is involved in drilling the wells, can lead to disruption of the commercial activities at the active burial region.

The landfill site may have a waste-in-place region and an active region. Gas collection wells may be placed in the waste in place region to allow the wells to tap into the gas that's produced by the decaying waste.

The disclosed system and method use the drone methane, or other trace gas, concentration over the surface as an analogue for where to place these wells. In some embodiments, wells may be placed based on an expected amount of waste that's in the landfill site and an expected amount of methane that a type of waste will generate. Wells may therefore be sized according to the expectation. However, expectations around waste decay, and the methane that is produced result in a wide amount of uncertainty, because the waste is not homogeneous. Waste is often heterogeneously put into a landfill. As a result, the distribution of the gas in the subsurface may be nothing like expected based on these predictions.

In some embodiments, wells may be drilled in and may not be optimally placed for that gas collection. In most cases, this placement of wells does not access all the gas and/or optimize the gas being accessed in an efficient manner. In many embodiments, the wells are not very deep and may be only a couple hundred feet maximum.

A better way to place those gas collection wells is shown in the disclosed system and method. In many cases, trace gas detected at the surface may be typically representative of what's going to be in the subsurface. By flying the drone and obtaining a detailed heat map as disclosed herein, a distribution of high concentration of methane may be determined. The disclosed system and method may then determine the location to drill and/or place wells. Additionally, the detected trace gas may be used to size the wells so that the size of equipment needed to cope with the expected gas flow may be determined before the well is installed.

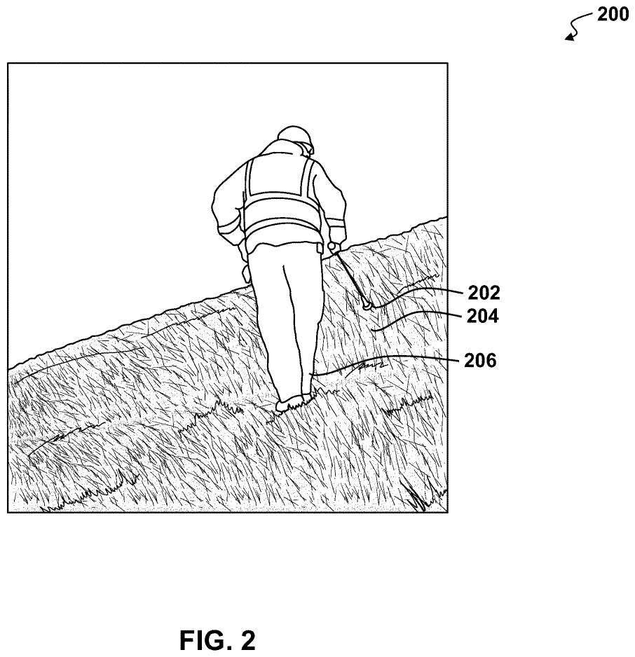

Ahead of the placement of any test wells to sample the subsurface, one method for obtaining an indication of the methane concentrations near the surface is to perform a manual walking survey 200 of a grassy region by a human operator 206, which may involve holding a methane sensor 202 extremely close to the surface 204 as shown in FIG. 2. The manual walking survey 200 is a potentially dangerous, time-consuming, and unpleasant job. The system and method disclosed herein replaces the need for a manual walking survey 200. At a minimum, the system will constrain the walking survey 200 to search areas where the exact ground location of the emission can then be confirmed.

One considerable expense when performing the walking survey is that the grass over the landfill surface needs to be mown to ensure that the walking survey can be performed. Given the varying topography, that gas wells may already be present, and the difficulty for a mower to access parts of the site, the mowing can be an expensive undertaking. Landfills may be quite hilly regions where tractors must go and mow the grass, which results in a sizeable expense for them to mow the grass before each walking survey.

With a drone in the disclosed system and method, the grass does not need to be mowed, and so this expense is saved. The system disclosed herein may fly just above the grass and negate the need for mowing. Furthermore, due to the ability to computer control the UAS, repeatable paths over the surface of the landfill can be undertaken over various time intervals, leading to consistent measurements, and enabling direct comparisons of the evolution of the emissions and near-surface methane distribution. The walking survey often does not follow the same path, leading to difficulties in evaluating the temporal evolution of the near-surface methane emissions.

Another issue with a manual walking survey is that it may put someone at risk, whereas the drone is completely autonomous and remote. The drone may be faster than a person conducting a walking survey. The drone may be safer than putting a person at risk walking over a landfill site.

The disclosed system and method using a drone is also consistent and repeatable. Walking surveys may typically follow a serpentine path that walks over the surface of the landfill. As with many human walking paths, it's by no means as tight as a computer controlled path can be. When repeat surveys are needed, the disclosed system and method are completely repeatable. Further, the disclosed system and method may be partially or completely automated. The disclosed system and method are safer, faster, and more efficient as compared to a walking survey.

The disclosed system and method for ascertaining where to put gas collection wells provides the following benefits. In the disclosed system and method, there is no need to prepare the grassy surface ahead of any surveys: the sensitivity of the drone-based measurement is such that it can be remote from the surface but still identify with extreme precision regions of enhanced methane and/or other trace gases. In the disclosed system and method, humans are not put at risk by deploying a system that can be deployed with a UAS. The UAS can be deployed over the surface on either visual line of sight (VLOS) or beyond visual line of site models (BVLOS), with appropriate regulatory and local operator approvals. The drone may fly a dense hatch pattern across the surface to provide a much more detailed coverage as compared to a walking survey as shown in FIG. 2.

The disclosed system and method avoid the cost of drilling multiple wells across the surface of the WIP region, wells which may eventually have to be capped or plugged. The disclosed system and method avoid the expense of running tools that measure methane concentrations in the subsurface. The disclosed system and method minimize drilling expenditure to only the wells that will be used in the gas collection process.

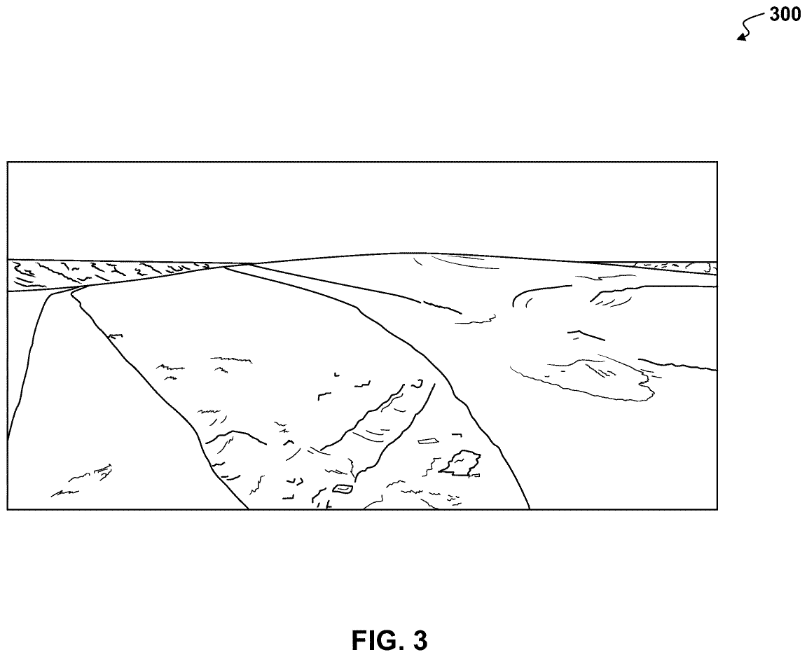

The process described below is unobtrusive and minimally invasive. It involves a number of steps. In a first step shown in FIG. 3, an aerial mapping survey of the landfill can yield high resolution orthomosaic maps and topographic models that can enable the measured methane enhancements to be placed in context of the physical location of the site 300. This information may already be available from the landfill operator as they have high resolution visual surveys for other reasons. FIG. 3 shows an orthomosaic image combined with a digital elevation model to effectively reconstruct the landfill in a digital format. FIG. 3 has been tilted away from, a top-down view such that the variation in the surface elevation of the site 300 can be seen.

The aerial survey may be conducted 1-10 meters above the ground. The disclosed system and method may use UAS terrain following capabilities to ensure that a consistent distance is kept as near as possible to the surface of the waste-in-place region. The distance above the ground may be varied based on any obstacles, the presence of trees or other objects, and the like. For example, where grass has not been mown, the drone may fly 1-2 meters above the ground while the drone may fly 0-1 meters above the ground where the grass has been mowed or grass is not otherwise present. In some embodiments, a drone operator may adjust the height of the drone relative to the surface. In some embodiments, the drone may maintain the same relative altitude during the entire flight. In other embodiments, the drone may use a global positioning system (GPS) or barometric pressure altitude to maintain the same altitude above sea level while relative altitude changes depending on the surface the drone is flying over. In some embodiments, the drone may include Light Detection and Ranging (LiDAR) to detect an exact relative altitude as well as GPS, trace gas sensor, and wind meter reading. Changes in altitude may be accounted for when determining a trace gas concentration over an area of the terrain.

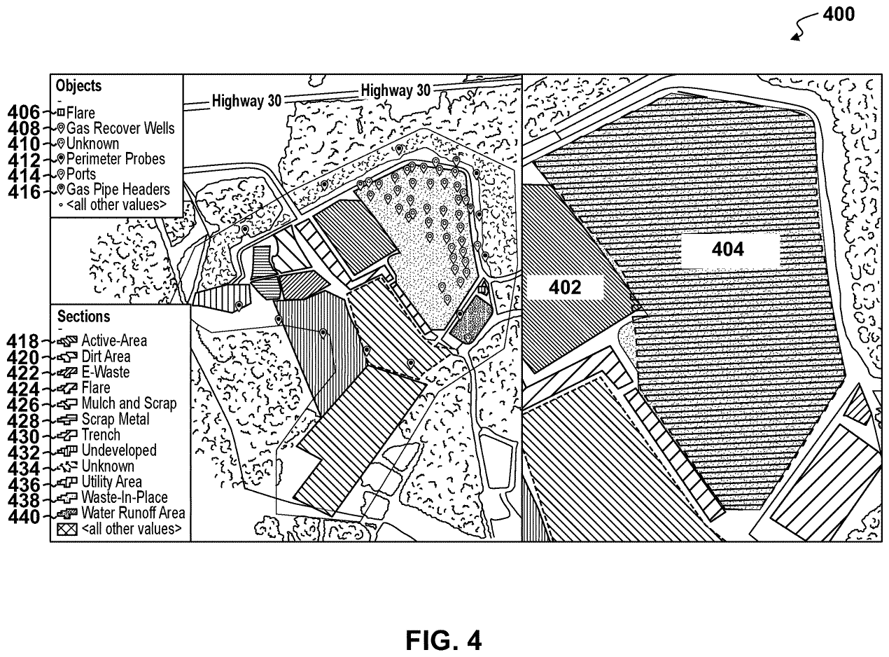

The second step shown in FIG. 4, is the identification 400 of the areas on the landfill site and the phase they are in, active area 402 or permanently capped (WIP) 404. The objects include flare 406, gas recovery wells 408, unknown objects 410, perimeter probes 412, ports 414, and gas pipe headers 416. The sections include active-area 418, dirt area 420, e-waste 422, flare 424, mulch and scrap 426, scrap metal 428, trench 430, undeveloped 432, unknown sections 434, utility area 436, waste-in-place 438, and water runoff area 440.

A coarse survey of the landfill site and surrounding area that enables a measurement of the background levels of methane and/or other trace gases. Many landfill sites will have a high level of methane due to releases from the active burial region. The disclosed highly sensitive and accurate sensor may be used to sample directly with no false positives. To ensure that the near-surface measurements of methane from the system accurately represent the subsurface, the disclosed system and method consider the methane that will be present due to the active burial region/working face. By encompassing the full three-dimensional nature of those emissions by deploying the system in and around the working face, the disclosed system and method may then remove these emissions from those that are a result of seepage or propagation from the subsurface on the waste-in-place section, which are those being targeted as representative of the subsurface methane distribution. Additionally, the disclosed system and method may also measure the emissions from any existing gas collection wells, or gas pipe headers, that have been previously placed on the waste-in-place region as they too can leak due to poor completions and lead to a non-representative subsurface distribution by contaminating the surface with their emissions.

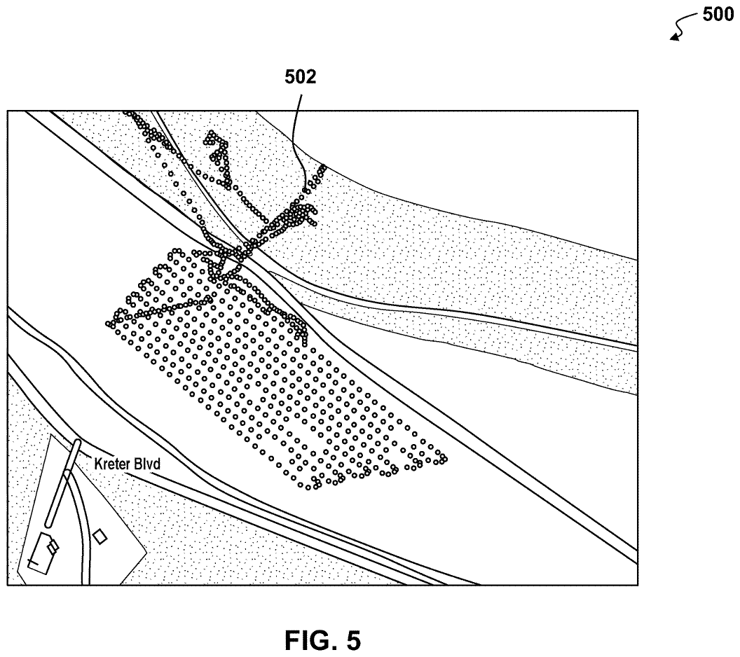

In a third step shown in FIG. 5, a survey is conducted of the methane enhancements across the surface of the landfill site 500. FIG. 5 specifically shows the geolocated measurements as points 502 along the flight path. The sampling density and pattern of the points 502 is a function of the drone flight velocity and sampling frequency. These regions may be selected for the gas collection wells. In such cases where there are enhancements on or near an existing well, it can trigger an inspection or maintenance procedure. The system and method may also be used for existing wells. In one embodiment, if an existing well shows as ‘hot’ then it may need maintenance. To ensure that the measurements made near the surface are as representative as possible, the disclosed system and method may use the terrain-following capabilities of the UAS. The disclosed system and method may also ensure that that ‘no-fly zones’, for example, other surface equipment, trees, etc., may also be excluded from the flight paths to ensure safe operations, by having them pre-programmed into the UAS flight paths. The points 502 in FIG. 5 depict the flight path and location of the trace gas sensor during each methane and/or other trace gas sensor reading. Each methane reading location may also include information on the position of the sensor (including absolute elevation and/or elevation about the ground surface) and wind direction and speed.

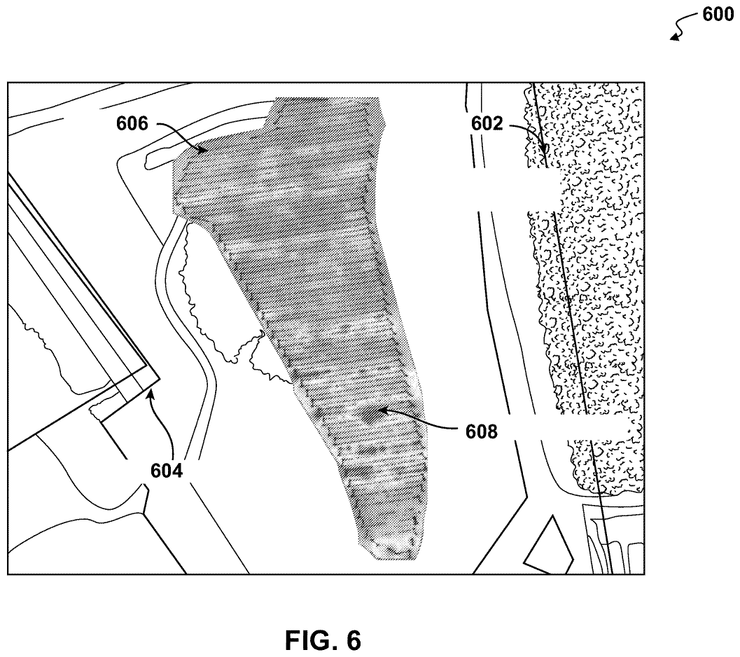

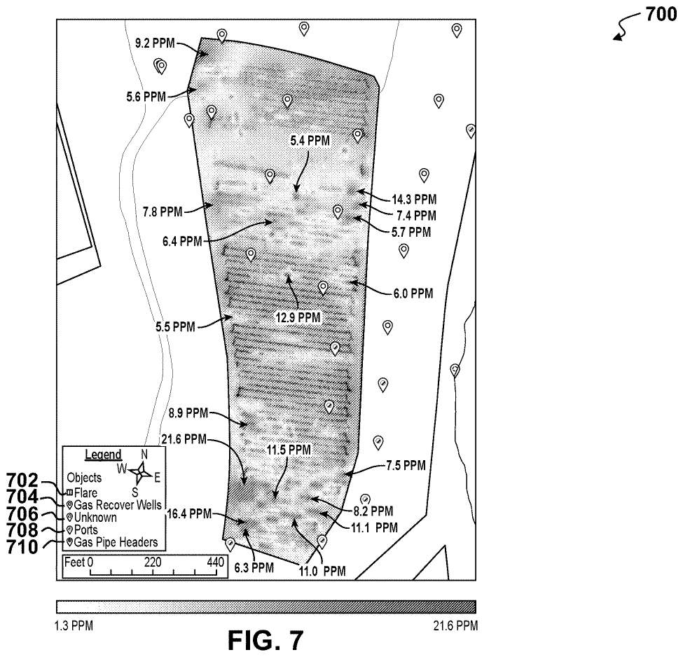

FIG. 6 highlights the methane enhancement results of a dense survey showing the anomalous methane regions over the WIP for one landfill site 600. The landfill site 600 includes a site outline 602, a detected emissions outline 604, a flight path 606, and detected emissions 608 in the region of the flight path 606. Hotspot regions may be selected for the gas collection wells. In such cases where there are enhancements on or near an existing well, it can trigger an inspection or maintenance procedure. The system and method may also be used for existing wells. In one embodiment, if an existing well shows as ‘hot’ then it may need maintenance. Darker areas in FIG. 6, such as detected emissions 608, indicate areas of higher methane concentration. A false-color image may be generated such that low methane enhancements are indicated by transparent-to-light yellow colors. The largest methane enhancements are indicated by red colors. Other colors and indicators are possible and contemplated to show areas of higher methane concentration. The methane enhancement results may be scaled and colored by emissions enhancement (parts per million (ppm) over the background as calculated) to better visualize the locations of any methane emissions. A scale bar is shown in FIG. 7. The detected emissions outline 604 is shown relative to the flight path 606. The drone may be flown until a decrease in background emissions is detected, which may be used to determine the boundary of the elevated emissions.

The detected emissions outline 604 is showing which equipment group area has enhancements. For example, the detected emissions outline 604 may include the waste-in-place area. The lower right corner outline may be the flare area. The left outline group may be the active area. These areas may be labeled once a report is generated. These equipment groups may be provided by onsite personnel or company.

FIGS. 7, 9, and 10 depict a two-dimensional ‘heatmap’ representative slice of the emissions captured at the time of flight. The sensor may not always be a downward facing camera, LiDAR, or reflectivity method, but a direct measure of the in-situ methane concentrations. The measurements may be affected by wind variability (which may be measured on location such as via an anemometer on the drone and/or a separate wind measurement system). Given the inherent variability and complexity of near surface wind flows, particularly in regions of complex topography, multiple passes may be aggregated to give more confidence in the specific location, concentrations/emission rate to allow for the variable winds and potential intermittency of the sources. The disclosed system and method may be a significantly more cost-effective operation than drilling multiple sample wells. The disclosed system and method leverage the automation capabilities of the drones and may use pre-programmed waypoints and flight sequences so that spatial invariance can be removed. The disclosed system and method may focus on the temporal evolution of the methane enhancements.

FIGS. 7-9 highlight the actual measured enhancements over a number of regions of the landfill, clearly indicating the regions of high concentration that would be considered for gas collection well placement. FIG. 7 is a top-down view of the measured methane enhancements 700 showing a peak concentration in the bottom left of 21.6 ppm. The whole survey shown in FIG. 7 may take no more than 30 minutes, which is a significant improvement in the time it would take to perform a preliminary walking survey (See FIG. 2), and drill multiple sample wells.

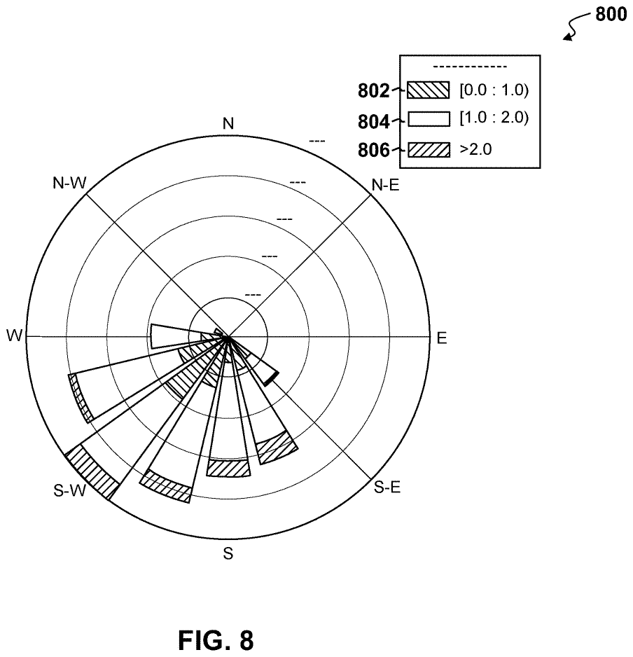

FIG. 7 is similar to FIG. 6 but is for a different detection survey. FIG. 7 includes objects including: flare 702, gas recovery wells 704, unknown objects 706, ports 708, and gas pipe headers 710. Additionally, FIG. 7 shows the range of methane enhancement (in PPM) with a legend with shading indicating the concentration of methane between 1.3 PPM and 21.6 PPM. Other levels in PPM of methane are possible and contemplated. A stationary, ground-based weather station may be used to measure the wind speed and direction at a representative location during the survey. The measured wind field, shown as a windrose diagram in FIG. 8, along with drone survey altitude, is used to provide context on the probable ground-location of fugitive methane leaks.

FIG. 8 highlights the variability of the wind during the measurement campaign, and why in this case it would be necessary to look at a few surveys to ensure the consistency of the location of the surface enhancements, as the wind shifts from South-West to South-East during the survey measurement period. FIG. 8 depicts a windrose 800 showing wind variability (in meters/second) during the methane measurements. Wind direction is indicated by the polar axes coordinates and the wind speed is shown in units of meters per second. Wind is measured in meters per second (m/s). Wind is shown as between 0.0 and 1.0 m/s 802; between 1.0 and 2.0 m/s 804; and greater than 2.0 m/s 806.

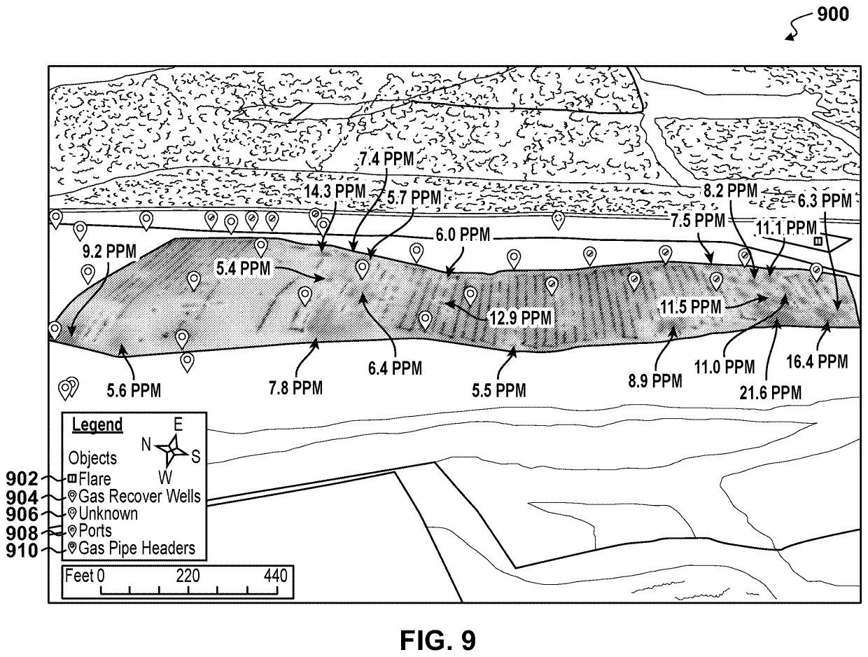

FIG. 9 is a side view (or tilted from top-down) that gives an indication of the relief or topography 900, a factor that may be important in ultimately determining the position of the gas collection wells. The topography 900 may include objects including flare 902, gas recovery wells 904, unknown objects 906, ports 908, and gas pipe headers 910. The gas collection wells may be easier to install on a flat or horizontal section, but the actual high concentration region may be located in a more sloped section, which will give a much higher volume of gas production even if the well construction is more complex, and thus may lead to a higher cost of well installation. However, this cost may be offset by the enhanced gas production. The methane gas concentrations in FIG. 9 are shown in parts per million (ppm).

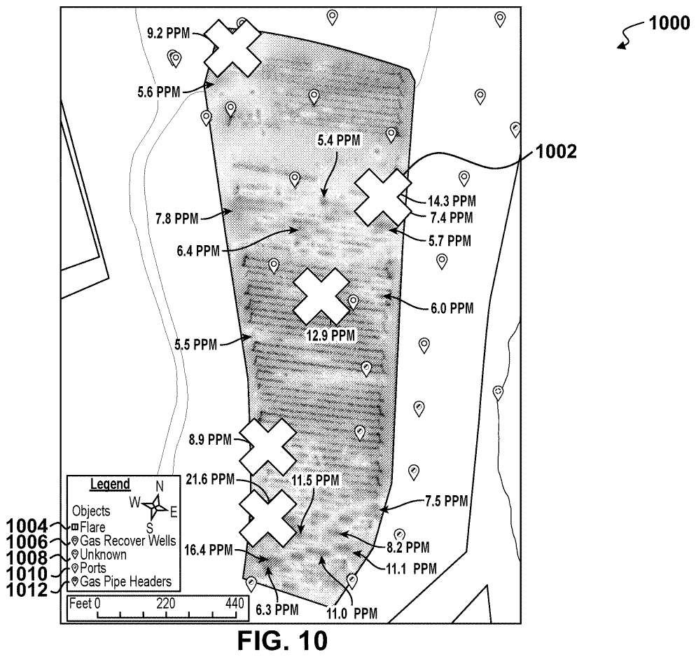

With the information contained within, the disclosed system and method may start to ascertain where the best place would be to place the gas collection wells. A first pass of the selection 1000 is shown by the placement of the X's 1002 in FIG. 10. The site may include objects including: flare 1004, gas recovery wells 1006, unknown objects 1008, ports 1010, and gas pipe headers 1012. The ultimate number of wells that would be needed is a function of the expected production rate, and the size of upgrading system that would be used on the site, as well as factoring the total cost of installation relative to the gas production rate. The well placements are put where the highest concentrations of methane are observed, once any gas that has been measured as originating from either previously installed wells or the active burial region working face has been removed from the totals. On a first pass, well placement is decided at the hotspot location(s) meeting or exceeding a concentration enhancement threshold.

Alternative methods to assess the position of placement of the gas collection wells that would lead to maximum or at least improved gas production volumes would be to use a sparser walking survey and place the wells based on the local concentration maxima seen during that walking survey or use pre-planned expected sub-surface gas concentration distribution and drill test wells to validate that the concentrations or gas distributions are as expected. Both would be more expensive and less practical than the invention described here.

There are conditions when the drone is surveying at an altitude that is high above ground level and/or the horizontal wind advection is large, meaning that the methane enhancement hotspots are displaced from their respective emission locations at ground level. In this scenario, the disclosed system and method can make an estimate of the distance traveled from the emission source location and translate (or shift) the hotspot backward along the prevailing wind direction to help constrain the well-placement locations. The disclosed system and method use a plume rise parameterization that is a function of drone altitude, methane gas density, temperature, pressure, horizontal wind speed, and estimated gas release characteristics of exit diameter and exit velocity. The disclosed system and method then solve the plume rise parameterization to give an estimate of the downwind distance traveled from ground level.

In other embodiments, the in-situ gas sensor may be placed on a ground-based robotic system. In other embodiments, the heat map may be generated using the calculated mass flow rate of trace gas information. In another embodiment, the sensor utilized may be a tunable diode laser absorption spectroscopy (TDLAS) sensor disposed on a robotic system that reflects off surfaces, generating a heat map of column-integrated concentration values. In this embodiment, the column-integrated concentration values may be multiplied by a wind speed value to generate a column-integrated mass flow rate over the given column height. This column-integrated mass flow rate may be used to generate a mass flow rate heat map to determine well placement.

The pattern flown may be any predefined and/or arbitrary pattern, including a raster pattern, spiral, and/or random motion. The pattern flown may also be modified based on data that is gathered in flight, such as gradient ascent, levy-flight, source-seeking, and the like. The modification of the pattern flown may result in a higher density of information in areas of higher concentration/mass flow and therefore higher value.

The sensor may be flown less than 20 m above the surface of interest, but higher than 0.5 meters above the ground. Other sensor heights are possible and contemplated.

In some embodiments, trace gas concentrations may be determined using satellite spectrometers and/or manned aircraft. In some embodiments, these methods may be used to determine areas for further inspection using the UAS and trace gas sensor as disclosed herein.

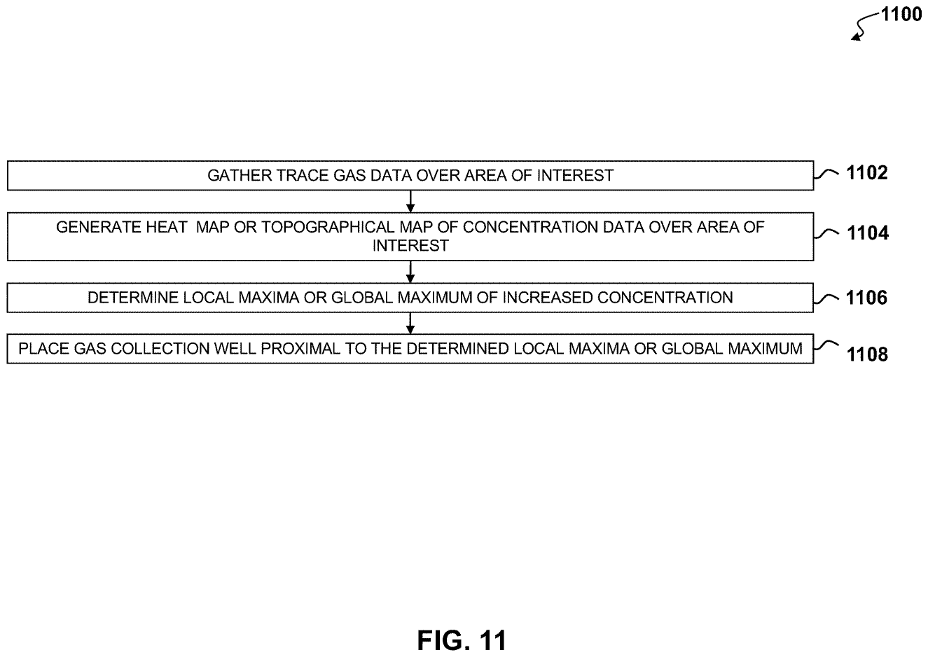

FIG. 11 depicts a high level flowchart of a method embodiment 1100 for determining the placement of a gas collection well based on gathered trace gas data. The method 1100 may include gathering trace gas data over an area of interest (step 1102). The method 1100 may then include generating a heat map or topographical map of concentration data of the area of interest (step 1104). The method 1100 may then include determining a local maxima or global maximum of increased concentration (step 1106). The method 1100 may then include placing a gas collection well proximal to the determined local maxima or global maximum (step 1108).

Additional method embodiments may include the measurement of emissions from previously installed gas collection wells to ensure that the heat map is more representative of the subsurface gas distribution as it will then only include the gas that has permeated from the subsurface. Additional method embodiments may include the measurement of emissions from the working face (and indeed all the active waste burial region) to ensure that the heat map is more representative of the subsurface gas distribution as it will then only include the gas that has permeated from the subsurface.

Additional method embodiments may include the use of UAS waypoint placement and automated flight capabilities to ensure that repeat flights over the surface of the waste-in-place site follow a consistent path each time. Additional method embodiments may include of a UAS terrain following capabilities to ensure that a consistent distance is kept as near as possible to the surface of the waste-in-place region.

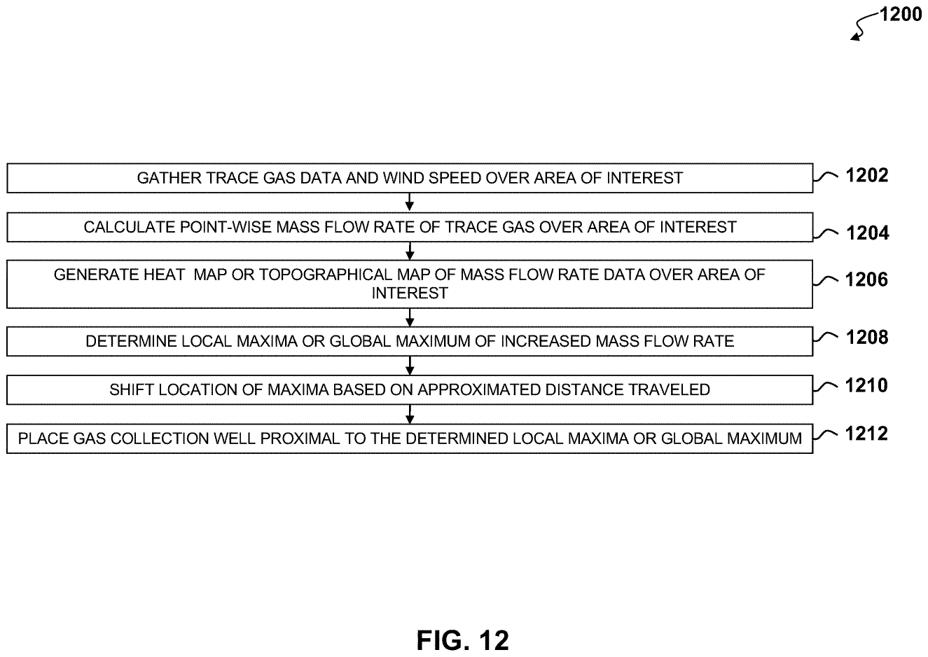

FIG. 12 depicts a high-level flowchart of a method embodiment 1200 for determining the placement of a gas collection well based on gathered trace gas data and wind speed. The method 1200 may include gathering trace gas data and wind speed over an area of interest (step 1202). The method 1200 may then include calculating a point-wide mass flow rate of a trace gas over an area of interest (step 1204). The method 1200 may then include generating a heat map or topographical map of the mass flow rate data over the area of interest (step 1206). The method 1200 may then include determining a local maxima or global maximum of increased mass flow rate (step 1208). The method 1200 may then include shifting a location of maxima based on an approximated distance traveled (step 1210). The method 1200 may then include placing a gas collection well proximal to the determined local maxima or global maximum (step 1212).

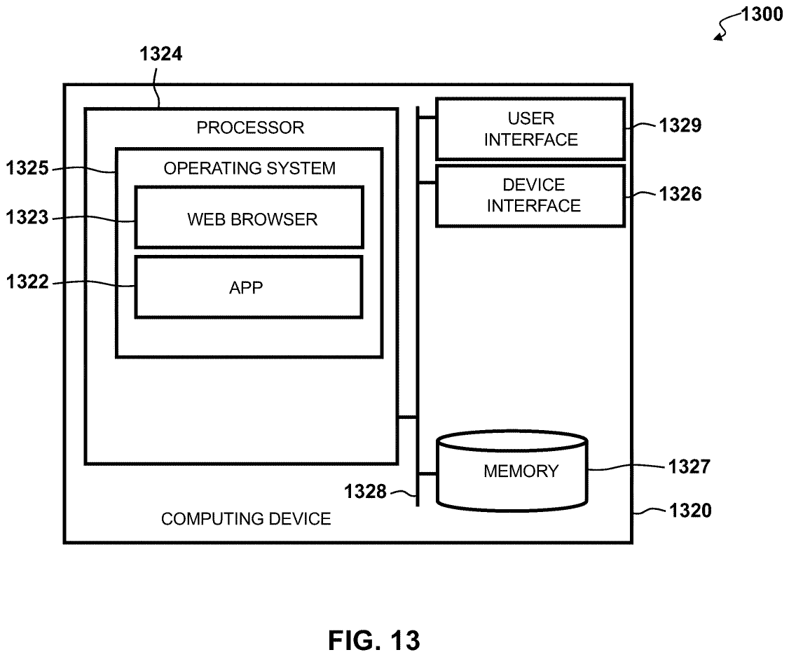

FIG. 13 illustrates an example of a top-level functional block diagram of a computing device embodiment 1300. The example operating environment is shown as a computing device 1320 comprising a processor 1324, such as a central processing unit (CPU), addressable memory 1327, an external device interface 1326, e.g., an optional universal serial bus port and related processing, and/or an Ethernet port and related processing, and an optional user interface 1329, e.g., an array of status lights and one or more toggle switches, and/or a display, and/or a keyboard and/or a pointer-mouse system and/or a touch screen. Optionally, the addressable memory may, for example, be: flash memory, eprom, and/or a disk drive or other hard drive. These elements may be in communication with one another via a data bus 1328. In some embodiments, via an operating system 1325 such as one supporting a web browser 1323 and applications 1322, the processor 1324 may be configured to execute steps of a process establishing a communication channel and processing according to the embodiments described above.

System embodiments include computing devices such as a server computing device, a buyer computing device, and a seller computing device, each comprising a processor and addressable memory and in electronic communication with each other. The embodiments provide a server computing device that may be configured to: register one or more buyer computing devices and associate each buyer computing device with a buyer profile; register one or more seller computing devices and associate each seller computing device with a seller profile; determine search results of one or more registered buyer computing devices matching one or more buyer criteria via a seller search component. The service computing device may then transmit a message from the registered seller computing device to a registered buyer computing device from the determined search results and provide access to the registered buyer computing device of a property from the one or more properties of the registered seller via a remote access component based on the transmitted message and the associated buyer computing device; and track movement of the registered buyer computing device in the accessed property via a viewer tracking component. Accordingly, the system may facilitate the tracking of buyers by the system and sellers once they are on the property and aid in the seller's search for finding buyers for their property. The figures described below provide more details about the implementation of the devices and how they may interact with each other using the disclosed technology.

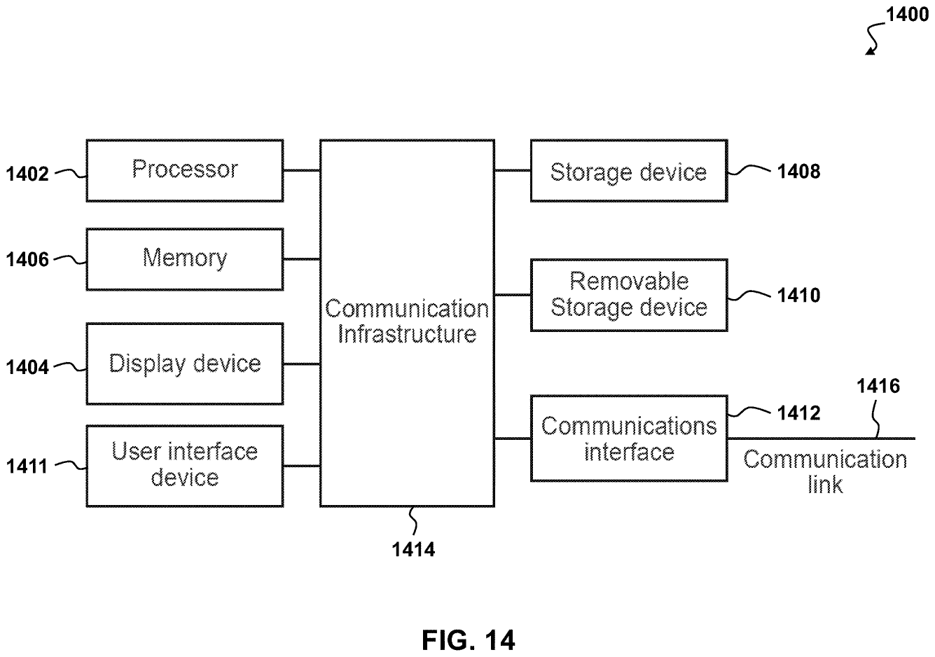

FIG. 14 is a high-level block diagram 1400 showing a computing system comprising a computer system useful for implementing an embodiment of the system and process, disclosed herein. Embodiments of the system may be implemented in different computing environments. The computer system includes one or more processors 1402, and can further include an electronic display device 1404 (e.g., for displaying graphics, text, and other data), a main memory 1406 (e.g., random access memory (RAM)), storage device 1408, a removable storage device 1410 (e.g., removable storage drive, a removable memory module, a magnetic tape drive, an optical disk drive, a computer readable medium having stored therein computer software and/or data), user interface device 1411 (e.g., keyboard, touch screen, keypad, pointing device), and a communication interface 1412 (e.g., modem, a network interface (such as an Ethernet card), a communications port, or a PCMCIA slot and card). The communication interface 1412 allows software and data to be transferred between the computer system and external devices. The system further includes a communications infrastructure 1414 (e.g., a communications bus, cross-over bar, or network) to which the aforementioned devices/modules are connected as shown.

Information transferred via communications interface 1414 may be in the form of signals such as electronic, electromagnetic, optical, or other signals capable of being received by communications interface 1414, via a communication link 1416 that carries signals and may be implemented using wire or cable, fiber optics, a phone line, a cellular/mobile phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 1412. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

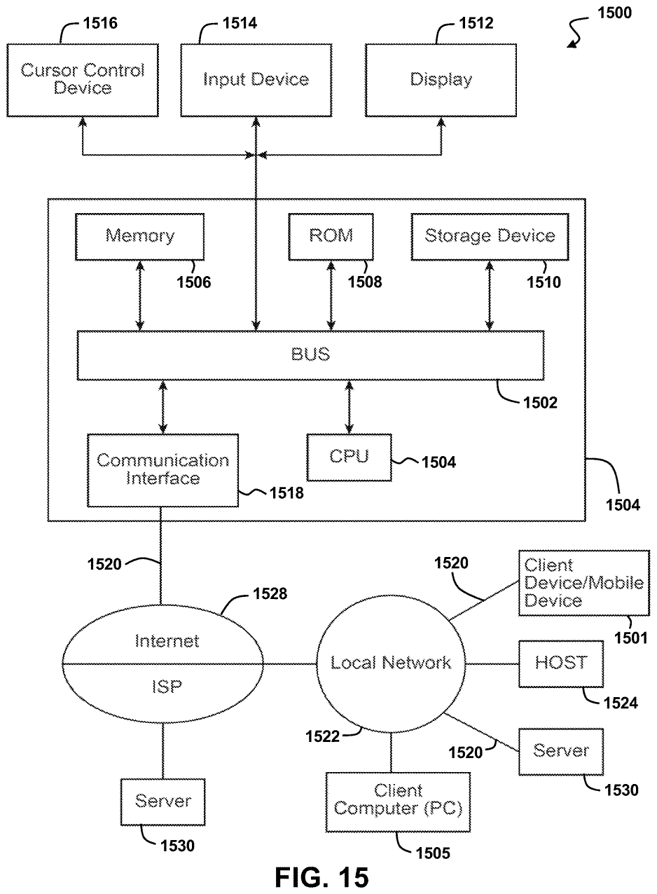

FIG. 15 shows a block diagram of an example system 1500 in which an embodiment may be implemented. The system 1500 includes one or more client devices 1501 such as consumer electronics devices, connected to one or more server computing systems 1530. A server 1530 includes a bus 1502 or other communication mechanism for communicating information, and a processor (CPU) 1504 coupled with the bus 1502 for processing information. The server 1530 also includes a main memory 1506, such as a random access memory (RAM) or other dynamic storage device, coupled to the bus 1502 for storing information and instructions to be executed by the processor 1504. The main memory 1506 also may be used for storing temporary variables or other intermediate information during execution or instructions to be executed by the processor 1504. The server computer system 1530 further includes a read only memory (ROM) 1508 or other static storage device coupled to the bus 1502 for storing static information and instructions for the processor 1504. A storage device 1510, such as a magnetic disk or optical disk, is provided and coupled to the bus 1502 for storing information and instructions. The bus 1502 may contain, for example, thirty-two address lines for addressing video memory or main memory 1506. The bus 1502 can also include, for example, a 32-bit data bus for transferring data between and among the components, such as the CPU 1504, the main memory 1506, video memory and the storage 1510. Alternatively, multiplex data/address lines may be used instead of separate data and address lines.

The server 1530 may be coupled via the bus 1502 to a display 1512 for displaying information to a computer user. An input device 1514, including alphanumeric and other keys, is coupled to the bus 1502 for communicating information and command selections to the processor 1504. Another type or user input device comprises cursor control 1516, such as a mouse, a trackball, or cursor direction keys for communicating direction information and command selections to the processor 1504 and for controlling cursor movement on the display 1512.

According to one embodiment, the functions are performed by the processor 1504 executing one or more sequences of one or more instructions contained in the main memory 1506. Such instructions may be read into the main memory 1506 from another computer-readable medium, such as the storage device 1510. Execution of the sequences of instructions contained in the main memory 1506 causes the processor 1504 to perform the process steps described herein. One or more processors in a multi-processing arrangement may also be employed to execute the sequences of instructions contained in the main memory 1506. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1504 for execution. Such a medium may take many forms, including but not limited to, non-volatile media, volatile media, and transmission media. Non-volatile media includes, for example, optical or magnetic disks, such as the storage device 1510. Volatile media includes dynamic memory, such as the main memory 1506. Transmission media includes coaxial cables, copper wire and fiber optics, including the wires that comprise the bus 1502. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 1504 for execution. For example, the instructions may initially be carried on a magnetic disk of a remote computer. The remote computer can load the instructions into its dynamic memory and send the instructions over a telephone line using a modem. A modem local to the server 1530 can receive the data on the telephone line and use an infrared transmitter to convert the data to an infrared signal. An infrared detector coupled to the bus 1502 can receive the data carried in the infrared signal and place the data on the bus 1502. The bus 1502 carries the data to the main memory 1506, from which the processor 1504 retrieves and executes the instructions. The instructions received from the main memory 1506 may optionally be stored on the storage device 1510 either before or after execution by the processor 1504.

The server 1530 also includes a communication interface 1518 coupled to the bus 1502. The communication interface 1518 provides a two-way data communication coupling to a network link 1520 that is connected to the worldwide packet data communication network now commonly referred to as the Internet 1528. The Internet 1528 uses electrical, electromagnetic, or optical signals that carry digital data streams. The signals through the various networks and the signals on the network link 1520 and through the communication interface 1518, which carry the digital data to and from the server 1530, are exemplary forms or carrier waves transporting the information.

In another embodiment of the server 1530, interface 1518 is connected to a network 1522 via a communication link 1520. For example, the communication interface 1518 may be an integrated services digital network (ISDN) card or a modem to provide a data communication connection to a corresponding type of telephone line, which can comprise part of the network link 1520. As another example, the communication interface 1518 may be a local area network (LAN) card to provide a data communication connection to a compatible LAN. Wireless links may also be implemented. In any such implementation, the communication interface 1518 sends and receives electrical electromagnetic or optical signals that carry digital data streams representing various types of information.

The network link 1520 typically provides data communication through one or more networks to other data devices. For example, the network link 1520 may provide a connection through the local network 1522 to a host computer 1524 or to data equipment operated by an Internet Service Provider (ISP). The ISP in turn provides data communication services through the Internet 1528. The local network 1522 and the Internet 1528 both use electrical, electromagnetic, or optical signals that carry digital data streams. The signals through the various networks and the signals on the network link 1520 and through the communication interface 1518, which carry the digital data to and from the server 1530, are exemplary forms or carrier waves transporting the information.

The server 1530 can send/receive messages and data, including e-mail, program code, through the network, the network link 1520 and the communication interface 1518. Further, the communication interface 1518 can comprise a USB/Tuner and the network link 1520 may be an antenna or cable for connecting the server 1530 to a cable provider, satellite provider or other terrestrial transmission system for receiving messages, data, and program code from another source.

The example versions of the embodiments described herein may be implemented as logical operations in a distributed processing system such as the system 1500 including the servers 1530. The logical operations of the embodiments may be implemented as a sequence of steps executing in the server 1530, and as interconnected machine modules within the system 1500. The implementation is a matter of choice and can depend on performance of the system 1500 implementing the embodiments. As such, the logical operations constituting said example versions of the embodiments are referred to for e.g., as operations, steps, or modules.

Similar to a server 1530 described above, a client device 1501 can include a processor, memory, storage device, display, input device and communication interface (e.g., e-mail interface) for connecting the client device to the Internet 1528, the ISP, or LAN 1522, for communication with the servers 1530.

The system 1500 can further include computers (e.g., personal computers, computing nodes) 1505 operating in the same manner as client devices 1501, where a user can utilize one or more computers 1505 to manage data in the server 1530.

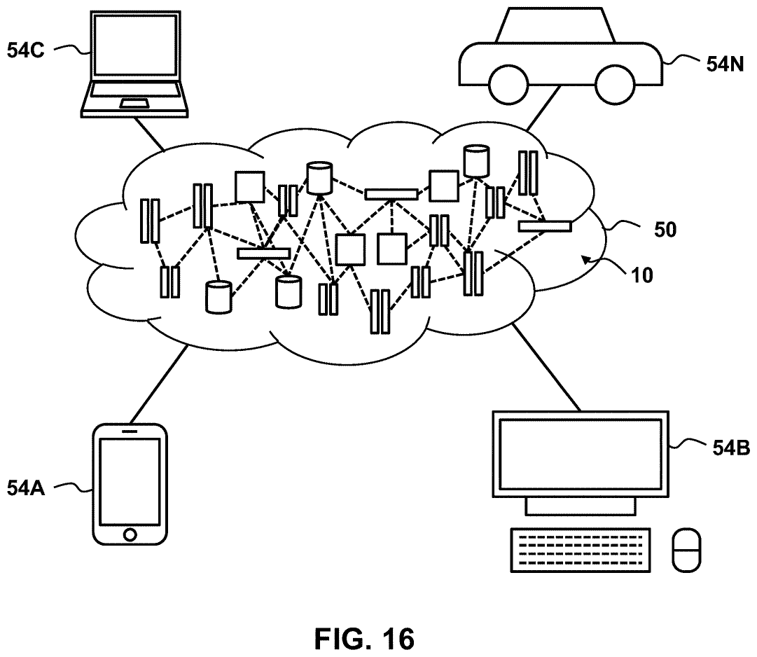

Referring now to FIG. 16, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or UAV system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 16 are intended to be illustrative only and that computing nodes 10 and cloud computing environment 50 can communicate with any type of computerized device over any type of network and/or network addressable connection (e.g., using a web browser).

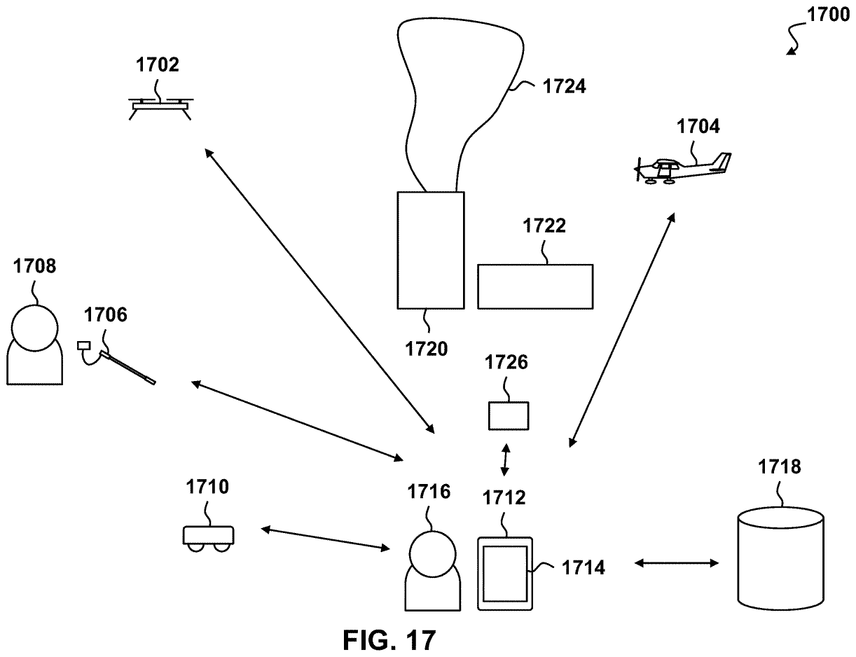

FIG. 17 depicts a system 1700 for detecting trace gases, according to one embodiment. The system may include one or more trace gas sensors located in one or more vehicles 1702, 1704, 1706, 1710. The one or more trace gas sensors may detect elevated trace gas concentrations from one or more potential gas sources 1720, 1722, such as a holding tank, pipeline, or the like. The potential gas sources 1720, 1722 may be part of a large facility, a small facility, or any location. The potential gas sources 1720, 1722 may be clustered and/or disposed distal from one another. The one or more trace gas sensors may be used to detect and quantify leaks of toxic gases, e.g., hydrogen disulfide, or environmentally damaging gases, e.g., methane, carbon dioxide, sulfur dioxide) in a variety of industrial and environmental contexts. Detection and quantification of these leaks are of interest to a variety of industrial operations, such as oil and gas, chemical production, and painting. Detection and quantification of leaks are also of value to environmental regulators for assessing compliance and for mitigating environmental and safety risks. In some embodiments, the at least one trace gas sensor may be configured to detect methane. In other embodiments, the at least one trace gas sensor may be configured to detect sulfur oxide, such as SO, SO₂, SO₃, S₇O₂, S₆O₂, S₂O₂, and the like. A trace gas leak 1724 may be present in a potential gas source 1720. The one or more trace gas sensors may be used to identify the trace gas leak 1724 and/or the source 1720 of the trace gas leak 1724 so that corrective action may be taken.

The one or more vehicles 1702, 1704, 1706, 1710 may include an unmanned aerial vehicle (UAV) 1702, an aerial vehicle 1704, a handheld device 1706, and a ground vehicle 1710. In some embodiments, the UAV 1702 may be a quadcopter or other device capable of hovering, making sharp turns, and the like. In other embodiments, the UAV 1702 may be a winged aerial vehicle capable of extended flight time between missions. The UAV 1702 may be autonomous or semi-autonomous in some embodiments. In other embodiments, the UAV 1702 may be manually controlled by a user. The aerial vehicle 1704 may be a manned vehicle in some embodiments. The handheld device 1706 may be any device having one or more trace gas sensors operated by a user 1708. In one embodiment, the handheld device 1706 may have an extension for keeping the one or more trace gas sensors at a distance from the user 1708. The ground vehicle 1710 may have wheels, tracks, and/or treads in one embodiment. In other embodiments, the ground vehicle 1710 may be a legged robot. In some embodiments, the ground vehicle 1710 may be used as a base station for one or more UAVs 1702. In some embodiments, one or more aerial devices, such as the UAV 1702, a balloon, or the like, may be tethered to the ground vehicle 1710. In some embodiments, one or more trace gas sensors may be located in one or more stationary monitoring devices 1726. One or more stationary monitoring devices may be located proximate to one or more potential gas sources 1720, 1722. In some embodiments, the one or more stationary monitoring devices may be relocated.

One or more vehicles 1702, 1704, 1706, 1710 and/or stationary monitoring devices 1726 may transmit data including trace gas data to a ground control station (GCS) 1712. The GCS may include a display 1714 for displaying the trace gas concentrations to a GCS user 1716. The GCS user 1716 may be able to take corrective action if a gas leak 1724 is detected, such as by ordering a repair of the source 1720 of the trace gas leak. The GCS user 1716 may be able to control movement of one or more vehicles 1702, 1704, 1706, 1710 in order to confirm the presence of a trace gas leak in some embodiments.

In some embodiments, the GCS 1712 may transmit data to a cloud server 1718. In some embodiments, the cloud server 1718 may perform additional processing on the data. In some embodiments, the cloud server 1718 may provide third-party data to the GCS 1712, such as wind speed, temperature, pressure, weather data, or the like.

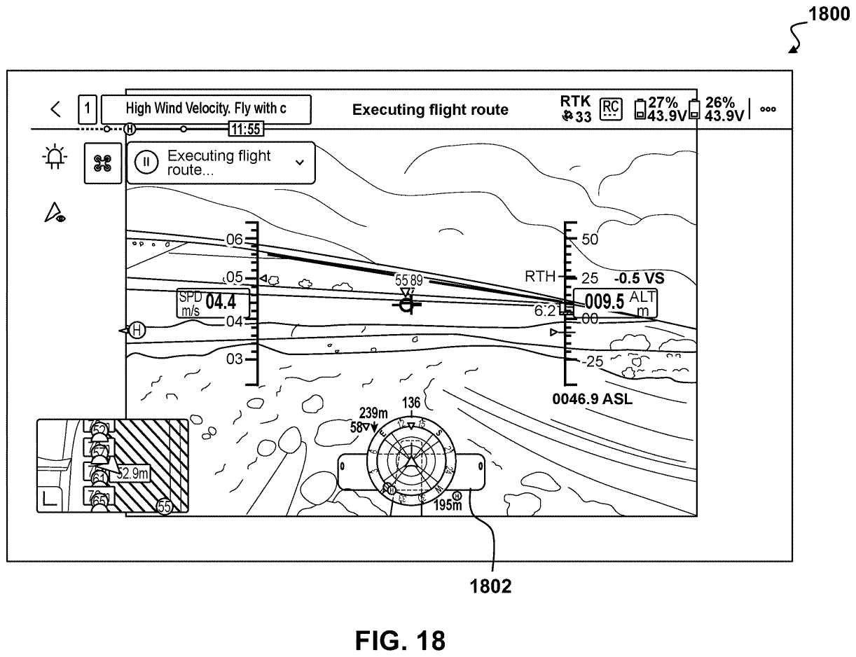

FIG. 18 depicts a field of view of an unmanned aerial system (UAS) flying over a landfill. The UAS may include a drone having a sensor 1802 to detect methane, carbon dioxide, and/or other trace gas concentrations. The UAS may also have a LiDAR or other systems to determine the elevation of the drone above the ground surface. In some embodiments, the drone may record video so that potential well sites may be visually referenced.

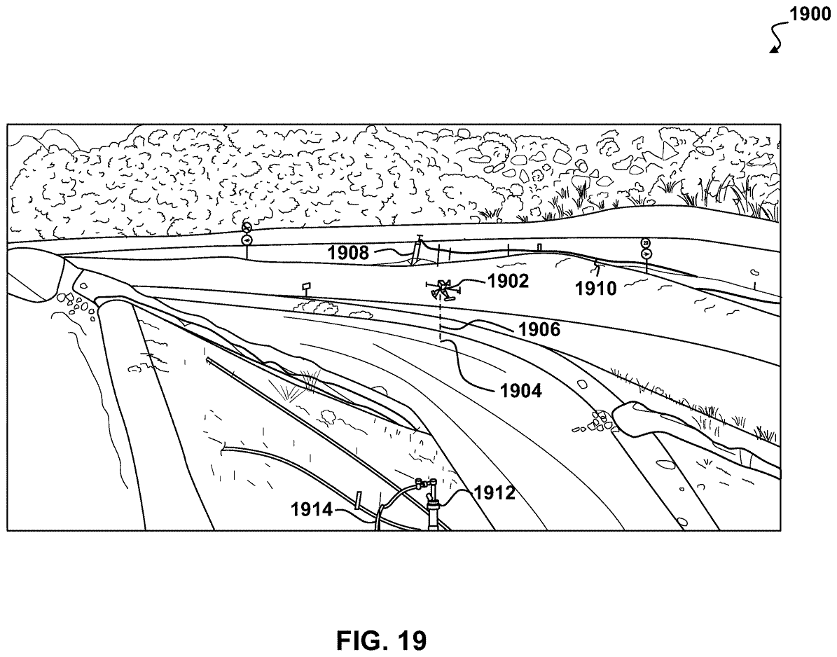

FIG. 19 depicts the UAS of FIG. 18 flying over the landfill 1900 with gas collection wells installed. The UAS 1902 may fly at a distance 1906, shown in a dashed line, between 1 and 10 meters above the surface 1904 of the landfill 1900. Other distances are possible and contemplated. Gas collection wells 1908, 1912 are shown with piping 1910, 1914 to transport methane, carbon dioxide, and/or other trace gases to another location for collection, use, power generation, and the like.

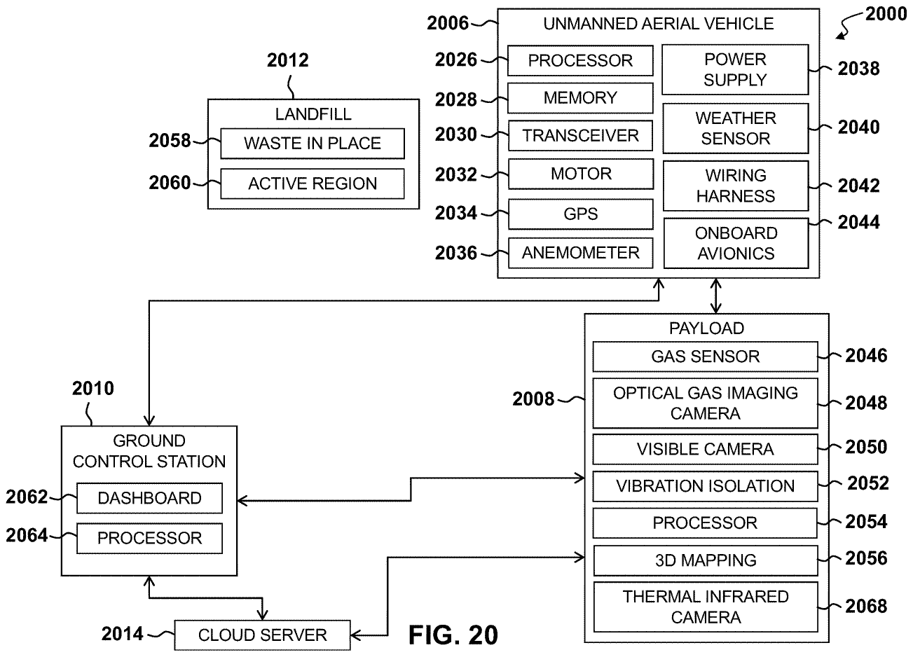

FIG. 20 depicts a high-level block diagram of an unmanned aerial system (UAS) trace gas detection system 2000. The system 2000 may include an unmanned aerial vehicle (UAV) 2006; a payload 2008 connected to the UAV 2006; a ground control station (GCS) 2010 that receives payload data from the payload 2008 relating to the landfill 2012; and a cloud server 2014 that may receive payload data from the GCS 2010 and/or the payload 2008.

The dashboard 2062 may include a display, user interface, processor, addressable memory, or the like. The dashboard 2062 may provide an ability to view and/or analyze data from the payload 2008 and/or UAV 2006. In some embodiments, the dashboard 2062 may be a dashboard with real-time data analysis and reporting. In other embodiments, the dashboard 2062 may be remote from the UAV 2006.

The UAV 2006 may include a processor 2026 in communication with addressable memory 2028, a transceiver 2030, one or more motors 2032, a global positioning system (GPS) 2034, an anemometer 2036, a power supply 2038, a weather sensor 2040, a wiring harness 2042, and/or onboard avionics 2044. The processor 2026 may store a flight plan, control the vehicle via interaction with the GCS 2010, control the vehicle via semi-autonomous control, and/or control the vehicle via autonomous control. The processor may also process payload data from the payload 2008 in some embodiments.

The disclosed UAV 2006 may be outfitted for the emissions sensor payload 2008; may have a flight time of greater than 1 hour; may have an emissions monitoring UAS payload 2008 with associated real-time data analysis algorithms; and/or may be fully autonomous. The UAV 2006 may be a standard UAS; have an endurance of about 120 minutes; may be vertical take-off and landing (VTOL) capable; may have a range of about 80 km; may have mission profiles for precision flights and long-linear; and/or may have a cruising altitude greater than 15 feet. Many inspection scenarios may require flight at low altitude and the expanded flight envelope for multi-rotor systems can accomplish this while fixed-wing systems are typically operated at 200-300 feet Above Ground Level (AGL).

In some embodiments, the UAV 2006 may be a multi-rotor, a transition vehicle, and/or a fixed-wind vehicle. A transition vehicle is a hybrid system that includes propellers for vertical takeoff and landing built around a fixed-wing platform for gliding over distance. A fixed-wing vehicle may offer the highest range for any platform, offering maximum flight times of 5 hours (~300 km linear range), but may require a far greater cruising altitude than other vehicles. The UAV 2006 may be a VTOL system for launch and/or recovery; may have a desired range of at least 80 km, which may increase with new technology; may have base station recharging capabilities, such as rapid recharging from a power system; may have a capability for low-altitude operations; and/or may have an autonomous hazard detection & avoidance system, such as via onboard avionics 2044. The disclosed system may provide capabilities to autonomously operate from a GCS 2010 and routinely inspect landfills 2012 including waste-in-place 2058 and active regions 2060.

The VTOL-capable UAV 2006 may be capable of autonomous launch and recovery. In some embodiments, the UAV 2006 may hover in place for data capture from a fixed point at different viewing angles and operations across a wide range of altitudes for high-resolution inspection.

In one embodiment, the UAV 2006 may have external dimensions of about 26 inches by 26 inches without propellers; an unladen weight of about 6.5 kg; a payload capacity of about 1.5 kg; a max flight time of up to 2 hours; a recharge time of about 1 hour for a 50% mission flight time; a max speed of about 19 meters per second (45 mph); a max distance of about 80 km; and/or a charge time of about 1 hour. The UAV 2006 may utilize lithium-ion batteries within the flight frame to increase flight time. Alternate dimensions and specifications of the UAV 2006 are possible and contemplated.

The transceiver 2030 may be in communication with the payload 2008, the GCS 2010, and/or the cloud server 2014. In some embodiments, the transceiver 2030 may be a transmitter or other communication device or system.

The one or more motors 2032 may drive one or more propellers. In some embodiments, there may be four motors in a quadcopter-style UAV 2006 configuration.

The GPS 2034 may determine the position of the UAV 2006 for following a flight plan, determining a location of the infrastructure 2012, and/or for including in the payload data from the payload 2008.

The anemometer 2036 may be used to measure a wind speed proximate to the UAV 2006 location and/or payload data. The wind speed measurement from the anemometer may be used with the payload data to determine a source location of an elevated ambient gas concentration. In some embodiments, the system 2000 may use wind speed from a third party source, an external anemometer, another UAV having an anemometer, and/or wind forecasts.

The power supply 2038 may be used to provide power to the motors 2032, payload 2008, and/or other systems and components of the UAV 2006. The mission distance and/or flight time may be limited by the available power of the power supply 2038.

The wiring harness 2042 may provide for power and/or data transmission on the UAV 2006 and/or payload 2008.

Onboard Avionics 2044 may be used to determine the UAV 2006 location, orientation, and the like. In some embodiments, the onboard avionics may be used as part of a sense and avoid system to avoid obstacles and/or infrastructure during the flight missions. The sense and avoid system may utilize autonomous flight path retasking (hazard avoidance).

The payload 2008 may include one or more gas sensors 2046, an optical gas imaging (OGI) camera 2048, a visible camera 2050, vibration isolation 2052, a processor 2054 having addressable memory, and/or a light detection and ranging (LIDAR) sensor. While various components are shown and described, it is contemplated that the elements of the UAV 2006 and payload 2008 may be interchanged and/or duplicated based on the needs of the system. For example, the UAV 2006 location from the GPS 2034 may be sent to the payload 2008 for inclusion in the payload data in some embodiments. In other embodiments, the payload 2008 may include a separate or redundant GPS or location sensor. In some embodiments, payload 2008 may include a methane (CH₄) and/or carbon dioxide (CO₂) sensor, such as the SeekIR sensor by SeekOps Inc. of Austin, Texas. The processor 2054 may perform onboard processing for data fusion and/or transmission. The processor 2054 requirements may be based upon required computing power, communication ports, power consumption, size, and the like. In some embodiments, the processor 2054 may be used to determine methane concentration in a landfill 2012. In some embodiments, the payload processor 2054, GCS processor 2064, and/or cloud server 2014 may be configured to determine methane concentrations in the landfill 2012. In some embodiments, the payload processor 2054 may be configured to fuse data from two or more sensors 2046, 2048, 2050, 2056, 2068 of the payload 2008.

The gas sensor 2046 may be used to determine an elevated methane, carbon dioxide, and/or other trace gas concentration. Elevated trace gas concentrations may be used to determine the location of a well in the landfill 2012.

The OGI camera 2048 may be used to detect a presence of certain gasses such as propane (C₃H₈), methane (CH₄), ethylene (C₂H₄), and the like. In one embodiment, the OGI camera may be the Ventus OGI by Sierra-Olympic Technologies, Inc, of Hood River, Oregon. In some embodiments, the OGI camera may be an infrared imager, such as an infrared imager tuned to mid-wavelength infrared (MWIR) or long-wavelength infrared (LWIR) for generating optical gas imaging (OGI) camera (2048) data.

The visible camera 2050 may be an RGB camera. The visible camera 2050 may provide visual context for the other payload data generated by the payload 2008. For example, the UAV mission may be surveying an active region 2060 of a landfill 2012. If elevated methane gas is detected by the gas sensor 2046 and/or OGI camera 2048, then a video feed or still image from the visible camera 2050 may allow an operator, or a machine-learning computer, to determine the placement of a well in that location with consideration of the topography of the region.

The payload 2008 may include vibration isolation 2052 in some embodiments to increase the accuracy of payload data, reduce errors, avoid damage to the components of the payload 2008, or the like. The combined payload 2008 weight and power consumption may be within set aircraft specifications. The vibration isolation 2052 may be a housing that protects all the sensors 2046, 2048, 2050, 2056, 2068 from light shock and dust and ensures proper vibration isolation.

The payload 2008 may include a LIDAR. The LIDAR may be used to image a ground location and to determine the altitude of the UAV 2006 and gas sensor 2046 above the ground surface of the landfill 2012.

The GCS 2010 may include a dashboard 2062 and a processor 2064 in communication with addressable memory. Payload data may be streamed from the payload 2008 to the GCS 2010 in real-time or near real-time. The payload data may include gas emissions data. The Dashboard 2062 may be used to display payload data from the payload 2008 and/or UAV 2006.

The GCS 2010 may be used to display real-time processed data on the dashboard 2062. The dashboard 2062 may provide live visual video for data context. In some embodiments, the dashboard 2062 may be a web-hosted Dashboard for data visualization with one or more of the following features: Displaying the UAV's 2006 current position and status; the ability to display raw payload values from each sensor (e.g., 4 in total); and the ability to display real-time processed data in an easy to interpret form. Data Analytics Algorithms may process the emissions data in near-real-time for view on the dashboard 2062. Data analysis algorithms for the emissions payload may provide a data visualization dashboard for real-time data; and a unique data presentation dashboard to rapidly display actionable information in real-time. This increased data and actionable steps provide increased emissions monitoring efficacy using the developed payload 2008.

In some embodiments, the dashboard 2062 may provide real-time visualization and presentation research; provide information that is useful for display and logging; OGI real-time processing algorithms; CH₄+CO₂ real-time processing algorithms; dynamic re-tasking; automatically ingesting and storing data received at the GCS 2010; algorithms that can be autonomously on the backend of the dashboard; moving and storing data in the cloud server 2014; real-time video data overlay; user access and/or security; restricting data and portal access based on credentials; and/or machine vision/learning.

The cloud server 2014 may be in communication with the GCS 2010, payload 2008, and/or UAV 2006. The cloud server 2014 may perform additional processing on the payload data, store the payload data in at least one database, compare payload data to historical payload data, or the like.

A system may include a processor (2026, 2054, 2064) having addressable memory. The steps disclosed herein may be performed on a processor 2064 of the ground control station 2010, a processor of the cloud server 2014, a processor 2026 of the UAV 2006, and/or a processor 2054 of the payload 2008. Every step disclosed herein may be performed by a same processor, in some embodiments. In other embodiments, steps may be performed by different processors. For example, the processor 2026 of the UAV 2006 may be used to control the UAV on a flight path determined by the processor 2064 of the ground control station 2010, the processor 2054 of the payload 2008 may be used to process trace gas data from the gas sensor 2046, and further analysis of the trace gas data may be performed by a processor of the cloud server 2014. Other processor divisions are possible and contemplated.

The processor may be configured to determine a flight path for an unmanned aerial vehicle (UAV) 2006 over at least a portion of a landfill 2012. The processor may also be configured to receive trace gas emissions, via a gas sensor 2046 of the UAV 2006, in the region of the flight path over the portion of the landfill 2012. The processor may also be configured to receive wind measurements corresponding to the received trace gas emissions. The processor may also be configured to determine one or more regions of elevated trace gas emissions in the landfill 2012 based on at least one of: the received trace gas emissions, the received wind measurements, and an elevation of the UAV 2006 above a ground surface of the landfill 2012 during the flight path. The elevation of the UAV 2006 above the ground may be used to determine a back trajectory of the trace gas emissions source as disclosed herein. The processor may also be configured to determine a placement of one or more gas collection wells in the landfill 2012 based on the determined one or more regions of elevated trace gas emissions. The one or more gas collection wells may be placed proximate the areas of elevated trace gas emissions, such as methane. The placement of the one or more gas collection wells may also consider the topography of the site as gas collection wells on a sloped surface may be more expensive than gas collection wells on a flatter surface.

The gas sensor 2046 may be configured to detect one or more of: methane and carbon dioxide. The elevation of the UAV 2006 above the ground surface of the landfill 2012 may be kept at a consistent distance via a terrain following of the UAV 2006. In other embodiments, the elevation of the UAV 2006 may vary based on how close the UAV 2006 may safely fly relative to the ground (e.g., considering obstacles, areas with grass, areas without grass or that have been mowed, and other factors). The elevation of the UAV 2006 above the ground surface of the landfill 2012 may be between 1 meter and 10 meters. Other distances are possible and contemplated. The processor may be further configured to repeat the determined flight path for the UAV (UAV) 2006 to determine any changes in the determine one or more regions of elevated trace gas emissions in the landfill 2012 over time. The received wind measurements may be via an anemometer 2036 on the UAV 2006. In other embodiments, the wind measurements may be from a fixed wind sensor on or near the landfill.

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention herein disclosed by way of examples should not be limited by the particular disclosed embodiments described above.

1. A method for determining a placement of a gas collection well based on gathered trace gas data, the method comprising:
gathering trace gas data over an area of interest using a sensor configured for deployment on a vehicle and configured to detect trace gas during the movement of the vehicle along a path to determine a boundary of elevated emissions;
generating at least one of: a heat map and a topographical map of gas concentration data based on the gathered trace gas data and the boundary, in the area of interest;
determining at least one of: a local maxima and a global maximum of increased gas concentration based on the at least one of: the heat map and the topographical map; and
determining placement of at least one gas collection well proximal to the determined at least one of: the local maxima and the global maximum.

2. The method of claim 1, wherein determining the boundary further comprises: determining the boundary based on detecting an increase in background trace gas levels followed by a decrease in background emissions.

3. The method of claim 1, further comprising:
removing trace gas emissions from an active burial region from those that are from a subsurface on a waste-in-place section, wherein the area of interest includes the active burial region and the waste-in-place section.

4. The method of claim 1, further comprising:
gathering trace gas data over at least one previously installed gas collection well.

5. The method of claim 1, further comprising:
gathering trace gas data from a working face of the area of interest to ensure that the generated heat map is more representative of a subsurface gas distribution.

6. The method of claim 1, further comprising:
gathering trace gas data from an active waste burial region to ensure that the generated heat map is more representative of a subsurface gas distribution.

7. The method of claim 1, further comprising:
placing one or more unmanned aerial system (UAS) waypoints over the area of interest; and
repeating one or more flights of the UAS over the area of interest using the placed one or more UAS waypoints to ensure that repeat flights following a consistent path.

8. The method of claim 1, further comprising:
utilizing a UAS system terrain following capability to ensure that a consistent distance is kept as near as possible to a surface of the area of interest.

9. A method for determining a placement of at least one gas collection well based on gathered trace gas data and wind speed, the method comprising:
gathering trace gas data and wind speed over an area of interest using a sensor configured for deployment on a vehicle and configured to detect trace gas during the movement of the vehicle along a path to determine a boundary of elevated emissions;
calculating a point-wide mass flow rate of a trace gas over the area of interest;
generating at least one of: a heat map and a topographical map of the mass flow rate data and said boundary, in the area of interest;
determining at least one of: a local maxima and a global maximum of increased mass flow rate based on the at least one of: the heat map and the topographical map; and
determining a placement of at least one gas collection well proximal to the determined at least one of: the local maxima and the global maximum.

10. The method of claim 9, wherein determining the boundary further comprises: determining the boundary based on detecting an increase in background trace gas levels followed by a decrease in background emissions.

11. The method of claim 9, further comprising:
removing trace gas emissions from an active burial region from those that are from a subsurface on a waste-in-place section, wherein the area of interest includes the active burial region and the waste-in-place section.

12. The method of claim 9, further comprising:
gathering trace gas data over at least one previously installed gas collection well.

13. The method of claim 9, further comprising:
gathering trace gas data from a working face of the area of interest to ensure that the generated heat map is more representative of a subsurface gas distribution.

14. The method of claim 9, further comprising:
gathering trace gas data from an active waste burial region to ensure that the generated heat map is more representative of a subsurface gas distribution.

15. The method of claim 9, further comprising:
placing one or more unmanned aerial system (UAS) waypoints over the area of interest; and
repeating one or more flights of the UAS over the area of interest using the placed one or more UAS waypoints to ensure that repeat flights following a consistent path.

16. The method of claim 9, further comprising:
utilizing a UAS system terrain following capability to ensure that a consistent distance is kept as near as possible to a surface of the area of interest.

17. A system comprising:
a processor having addressable memory, wherein the processor is configured to:
determine a flight path for an unmanned aerial vehicle (UAV) over at least a portion of a landfill;
receive trace gas emissions, via a gas sensor configured for deployment on the UAV, in the region of the flight path;
receive wind measurements corresponding to the received trace gas emissions;
determine one or more regions of elevated trace gas emissions in the landfill based on: the received trace gas emissions, the received wind measurements, and an elevation of the UAV above a ground surface of the landfill during the flight path;
determine a boundary of elevated emissions; and
determine a placement of at least one gas collection well in the landfill in the determined boundary of elevated emissions based on the determined one or more regions of elevated trace gas emissions.

18. The system of claim 17, wherein determining the boundary further comprises: determining the boundary based on detecting an increase in background trace gas levels followed by a decrease in background emissions.

19. The system of claim 17, wherein the gas sensor is configured to detect one or more of: methane and carbon dioxide.

20. The system of claim 17, wherein the elevation of the UAV above the ground surface of the landfill is kept at a consistent distance via a terrain following of the UAV.

21. The system of claim 17, wherein the elevation of the UAV above the ground surface of the landfill is between 1 meter and 10 meters.

22. The system of claim 17, wherein the processor is further configured to repeat the determined flight path for the UAV to determine any changes in the determine one or more regions of elevated trace gas emissions in the landfill over time.

23. The system of claim 15, wherein the received wind measurements are via an anemometer on the UAV.
